SEQUENCE LISTING (26 sequences)

SEQ ID NO: 1 - DNA, 63 bp, synthetic construct
  source 1..63: mol_type = other DNA
atggccttaccagtgaccgccttgctcctgccgctggccttgctgctccacgccgccaggccg

SEQ ID NO: 2 - protein, 21 aa, synthetic construct
MALPVTALLLPLALLLHAARP

SEQ ID NO: 3 - DNA, 744 bp, synthetic construct
  source 1..744: mol_type = other DNA
ctgcatgtaggctgtgctggaggattcgtctacagtcaatgtgaccttgtccttgaactcttgattgtagttagtataaatataagaaggataaatatttccgatccactcaaggccttgtccaggcctctgcttcacccagtttatccagtagttggtgaaggtgtagccagaagccttgcaggacagcttcacagccccaggcctcaccagttcagccccctgctgcagcttgacctgctcagcagcccgacatctgaggactctgcggtctattactgtacaagatccccttatggttacgacgagtatggtctggactactggggccaaggcaccacggtcaccgtctcctcaggtggtggcggttcaggcggaggtggctctggcggtggcggatcggacatcgagctcactcagtctccatcctccctgactgtgacagcaggagagaaggtcactatgaactgcaagtccagtcagagtctgttaaacagtagaaatcaaaagaactacttgacctggtaccagcagaaaccagggcagcctcctaaactgttgatatactgggcatccactagggaatctggggtccctgatcgcttcacaggcagtggatctggaacagatttcactctcaccatcagcagtgtgcaggctgaagacctggcagtttattactgtcagaatgattatgtttatccgctcacgttcggtgctgggaccaagctggaaataaaacgg

SEQ ID NO: 4 - protein, 248 aa, synthetic construct
LHVGCAGGFVYSQCDLVLELLIVVSINIRRINISDPLKALSRPLLHPVYPVVGEGVARSLAGQLHSPRPHQFSPLLQLDLLSSPTSEDSAVYYCTRSPYGYDEYGLDYWGQGTTVTVSSGGGGSGGGGSGGGGSDIELTQSPSSLTVTAGEKVTMNCKSSQSLLNSRNQKNYLTWYQQKPGQPPKLLIYWASTRESGVPDRFTGSGSGTDFTLTISSVQAEDLAVYYCQNDYVYPLTFGAGTKLEIKR

SEQ ID NO: 5 - DNA, 135 bp, synthetic construct
  source 1..135: mol_type = other DNA
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 6 - protein, 45 aa, synthetic construct
TTTPAPRPPTPAPTIASQPLSLRPEACRPAAGGAVHTRGLDFACD

SEQ ID NO: 7 - DNA, 204 bp, synthetic construct
  source 1..204: mol_type = other DNA
ttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 8 - protein, 68 aa, synthetic construct
FWVLVVVGGVLACYSLLVTVAFIIFWVRSKRSRLLHSDYMNMTPRRPGPTRKHYQPYAPPRDFAAYRS

SEQ ID NO: 9 - DNA, 126 bp, synthetic construct
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 10 - protein, 42 aa, synthetic construct
KRGRKKLLYIFKQPFMRPVQTTQEEDGCSCRFPEEEEGGCEL

SEQ ID NO: 11 - DNA, 336 bp, synthetic construct
  source 1..336: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 12 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 13 - RNA, 21 nt, synthetic construct
  source 1..21: mol_type = other RNA
acattctcgaagtcggtgacc

SEQ ID NO: 14 - RNA, 19 nt, synthetic construct
  source 1..19: mol_type = other RNA
ggtcacgactcgagaatgt

SEQ ID NO: 15 - RNA, 21 nt, synthetic construct
  source 1..21: mol_type = other RNA
ataaactccaggcctatgagg

SEQ ID NO: 16 - RNA, 19 nt, synthetic construct
  source 1..19: mol_type = other RNA
cctcatggctggagtttat

SEQ ID NO: 17 - RNA, 21 nt, synthetic construct
  source 1..21: mol_type = other RNA
ttctaatactacgttccgcat

SEQ ID NO: 18 - RNA, 19 nt, synthetic construct
  source 1..19: mol_type = other RNA
atgcggacgagtattagaa

SEQ ID NO: 19 - RNA, 141 nt, synthetic construct
  source 1..141: mol_type = other RNA
tggaggcttgctttgggctgtatgctgacattctcgaagtcggtgaccgttttggccactgactgacggtcacgactcgagaatgtcaggacacaaggccctttatcagcactcacatggaacaaatggcccgttaacgta

SEQ ID NO: 20 - RNA, 152 nt, synthetic construct
  source 1..152: mol_type = other RNA
tggaggcttgctttgggctgtatgctgataaactccaggcctatgagggttttggccactgactgaccctcatggctggagtttatcaggacacaaggccctttatcagcactcacatggaacaaatggcccgttaacgtagcatgcatgta

SEQ ID NO: 21 - RNA, 142 nt, synthetic construct
  source 1..142: mol_type = other RNA
tggaggcttgctttgggctgtatgctgttctaatactacgttccgcatggttttggccactgactgacatgcggacgagtattagaacaggacacaaggccctttatcagcactcacatggaacaaatggcccgttaacgta

SEQ ID NO: 22 - RNA, 20 nt, synthetic construct
  source 1..20: mol_type = other RNA
tacaggcagcaccagcgaag

SEQ ID NO: 23 - RNA, 20 nt, synthetic construct
  source 1..20: mol_type = other RNA
cttggcagttgagcacgcgc

SEQ ID NO: 24 - RNA, 100 nt, synthetic construct
  source 1..100: mol_type = other RNA
tacaggcagcaccagcgaaggttttagagctagaaatagcaagttaaaataaggctagtccgttatcaacttgaaaaagtggcaccgagtcggtgctttt

SEQ ID NO: 25 - RNA, 100 nt, synthetic construct
  source 1..100: mol_type = other RNA
cttggcagttgagcacgcgcgttttagagctagaaatagcaagttaaaataaggctagtccgttatcaacttgaaaaagtggcaccgagtcggtgctttt

SEQ ID NO: 26 - RNA, 100 nt, synthetic construct
  source 1..100: mol_type = other RNA
ggccgaaacctgatcctttagttttagagctagaaatagcaagttaaaataaggctagtccgttatcaacttgaaaaagtggcaccgagtcggtgctttt

(19) United States
(12) Patent Application Publication
(10) Pub. No.: US 2026/0028585 A1
(43) Pub. Date: Jan. 29, 2026

(54) METHOD FOR PREPARING ENHANCED CAR-T-CELLS BASED ON INTERFERING OR KNOCKING OUT HUMAN PCSK9 GENE AND APPLICATION THEREOF

(71) Applicant: Shenzhen University, Shenzhen (CN)

(72) Inventors: Shaojun XING, Shenzhen (CN); Bing LIU, Shenzhen (CN); Letong CAI, Shenzhen (CN); Yiwen YAN, Shenzhen (CN)

(73) Assignee: Shenzhen University, Shenzhen (CN)

(21) Appl. No.: 19/015,715

(22) Filed: Jan. 10, 2025

(30) Foreign Application Priority Data
Jul. 25, 2024 (CN) 202411003567.4

(51) Int. Cl. C12N 5/0783; A61K 40/11; A61K 40/31; A61K 40/42; C07K 16/28; C12N 9/22; C12N 15/11; C12N 15/113; C12N 15/86; C12N 15/87

(52) U.S. Cl. CPC C12N 5/0636; A61K 40/11; A61K 40/31; A61K 40/421; C07K 16/2827; C12N 9/22; C12N 15/111; C12N 15/1137; C12N 15/86; C12N 15/87; A61K 2239/13; A61K 2239/17; A61K 2239/21; A61K 2239/22; C12N 2310/141; C12N 2310/20; C12N 2740/15043; C12Y 304/21061

(57) ABSTRACT

A method for preparing enhanced CAR-T-cells based on interfering or knocking out the human PCSK9 gene and an application thereof are provided. The method includes performing the RNA interference (RNAi) for the human PCSK9 gene by the artificial microRNA (miRNA) to prepare the enhanced CAR-T or preparing the enhanced CAR-T by CRISPR-Cas9 gene-editing technology by using sgRNA to knock out the human PCSK9 gene. The method uses PCSK9 gene knockdown element or CRISPR/Cas9 technology to specifically knock down or knock out the expression of PCSK9 in CAR-T-cells, which effectively enhances the therapeutic effect of CAR-T-cells on malignant solid tumors, and can enhance the tumor therapeutic effect of immune checkpoint blockade therapy.

### CROSS REFERENCE TO THE RELATED APPLICATIONS

This application is based upon and claims priority to Chinese Patent Application No. 202411003567.4, filed on Jul. 25, 2024, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBZYGJ268_Sequence_Listing.xml, created on Dec. 20, 2024, and is 26,297 bytes in size.

### TECHNICAL FIELD

The invention relates to the field of CAR-T-cell preparation technology, in particular to a method for preparing enhanced CAR-T-cells based on interfering or knocking out a human PCSK9 gene and an application thereof.

### BACKGROUND

Chimeric antigen receptor T-cell (CAR-T) therapy is an immune cell therapy with a targeted function. Although CAR-T therapy has a significant therapeutic effect in hematological tumors, there are still great challenges in applying solid tumors. Studies have shown that there is heterogeneity in solid tumors, the tumor microenvironment is an immunosuppressive environment, and the function of CAR-T is inhibited by the tumor microenvironment. At present, immune checkpoint-blocking antibodies PD-1 and CTLA-4 have been used in the treatment of clinical tumor patients, but there are still many patients with low response to this immunotherapy. Therefore, it is essential to find a new method that can improve the activity of T-cells and enhance the immune checkpoint response.

Proproteinconvertase subtilisin/kexintype 9 (PCSK9), a tumor cell-derived proprotein convertase, can increase tumor immune escape, but the role of endogenous PCSK9 in immune cells against tumor immunity is not clear. Studies have found that PCSK9 expression is increased in tumor-infiltrating CD8+T-cells, knockout of PCSK9 in CD8+T-cells can significantly enhance its anti-tumor activity, prolong the survival cycle of experimental animals, and improve the therapeutic effect of immune checkpoint therapy.

In addition, due to the occurrence of graft-versus-host disease (GVHD) in allogeneic CAR-T, most of the current CAR-T is prepared by autologous T-cells. Clinically, tumor patients are often prone to lymphopenia after chemotherapy and radiotherapy, so the number of cells has become an important challenge for CAR-T treatment. Therefore, it is also important to adjust the CAR-T preparation process and reduce the demand for patients' peripheral blood mononuclear cells (PBMC).

B7H3, also known as CD276, is a type I transmembrane protein. The protein is overexpressed in a variety of malignant tumors and is rarely expressed in normal cells. The protein has an inhibitory effect on T-cells. At present, several CAR-T products targeting B7H3 have entered clinical trials, proving that the target has high specificity and safety.

The study of miRNA regulating gene expression is becoming more and more mature, and the technology of artificial miRNA silencing gene is also more and more used in tumor research, this method is based on the mammalian endogenous miRNA precursor as the basic skeleton, and replaces the miRNA sequence in the stem-loop structure with the artificial miRNA sequence partially complementary to the target gene mRNA, so that the generated artificial miRNA acts on the target gene, thereby inhibiting the expression of the target gene.

Clustered regularly interspaced short palindromic repeats (CRISPR)-CRISPR-associated protein 9 (Cas9) gene editing technology is to identify the target genome sequence through sgRNA and guide Cas9 nuclease to effectively cut the genome to form DNA double-strand breaks, and then realize gene knockout, knock-in, mutation, etc. through the cell's repair mechanism to achieve gene editing purposes, and has been used for the construction of CAR-T-cells. In CRISPR-Cas9 gene editing applications, the safe and effective delivery of the functional components of the CRISPR system to the target is a prerequisite for its role. The method of directly transferring ribonucleoprotein (RNP) formed by Cas9 and sgRNA into cells is safer and faster, with lower off-target effect and higher editing efficiency.

### SUMMARY

The purpose of this invention is to provide a method for preparing enhanced CAR-T-cells based on interfering or knocking out a human PCSK9 gene and an application thereof to improve the T-cell function by interfering or knocking out PCSK9 expression, and the knockout PCSK9 CAR-T preparation process is improved to reduce the demand for the number of patients' original PBMC.

In order to achieve the above purpose, the invention provides a miRNA fragment that interferes with an expression of the human PCSK9 gene, the miRNA fragment includes miRNA #603-guide sequence/passenger sequence, miRNA #792-guide sequence/passenger sequence;
a nucleotide sequence of miRNA #603-guide sequence is shown as SEQ ID NO: 13,
the nucleotide sequence of miRNA #603-passenger sequence is shown as SEQ ID NO: 14;
the nucleotide sequence of miRNA #792-guide sequence is shown in SEQ ID NO: 15,
the nucleotide sequence of miRNA #792-passenger sequence is shown in SEQ ID NO: 16;
using miR-155 as a skeleton, replacing a miRNA sequence in a miR-155 stem-loop structure with a miRNA fragment interfering with an expression of human PCSK9 gene to prepare an artificial miRNA for interfering with PCSK9 to prepare enhanced CAR-T;
the artificial miRNA includes miRNA #603 and miRNA #792, the nucleotide sequence of miRNA #603 is SEQ ID NO: 19, and the nucleotide sequence of miRNA #792 is SEQ ID NO: 20.

A sgRNA that knocks out a human PCSK9 gene, sgRNA includes sgRNA #1, sgRNA #6, and a spacer sequence of sgRNA #1 is SEQ ID NO: 22, the spacer sequence of sgRNA #6 is SEQ ID NO: 23;

after sgRNA is chemically synthesized into a ready-to-use sgRNA, the ready-to-use sgRNA is applied to knock out the human PCSK9 gene to prepare an enhanced CAR-T, the ready-to-use sgRNA includes ready-to-use sgRNA #1 and ready-to-use sgRNA #6, the nucleotide sequence of ready-to-use sgRNA #1 is shown in SEQ ID NO: 24, and the nucleotide sequence of ready-to-use sgRNA #6 is shown in SEQ ID NO: 25.

A method for preparing the enhanced CAR-T based on interfering with the expression of the human PCSK9 gene, using the artificial miRNA fragment containing the miRNA fragment interfering with the expression of the human PCSK9 gene, including the following steps:
S1, cloning a fusion gene fragment containing CD8a leader, B7H3 scFV, CD8 hinge region, CD28 transmembrane domain, 4-1BB intracellular domain and CD3ξ domain into a lentiviral backbone plasmid pCDH-EF1α to construct a recombinant lentiviral plasmid pCDH-EF1α-B7H3-CAR;
S2, linking the artificial miRNA into the recombinant lentiviral plasmid pCDH-EF1α-B7H3-CAR constructed in S1 to obtain a PCSK9 knockdown recombinant lentiviral plasmid pCDH-EF1α-miRNA-B7H3-CAR;
S3, co-transfecting pCDH-EF1α-miRNA-B7H3-CAR and a helper plasmid pMD2.G/pSPAX2 into 293T-cells for packaging to obtain a pCDH-EF1α-miRNA-B7H3-CAR lentivirus;
S4, using the pCDH-EF1α-miRNA-B7H3-CAR lentivirus obtained in S3 to infect activated T-cell in vitro to obtain a PCSK9 knockdown miR-B7H3-CAR-T-cell.

Preferably, in S1, the nucleotide sequence of CD8a leader is SEQ ID NO: 1; an amino acid sequence of CD8α leader is SEQ ID NO: 2;
the nucleotide sequence of B7H3 scFV is SEQ ID NO: 3;
the amino acid sequence of B7H3 scFV is SEQ ID NO: 4;
the nucleotide sequence of the CD8 hinge region is SEQ ID NO: 5;
the amino acid sequence of the CD8 hinge region is SEQ ID NO: 6;
the nucleotide sequence of the CD28 transmembrane domain is SEQ ID NO: 7;
the amino acid sequence of the CD28 transmembrane domain is SEQ ID NO: 8;
the nucleotide sequence of the 4-1BB intracellular domain is SEQ ID NO: 9;
the amino acid sequence of the 4-1BB intracellular domain is SEQ ID NO: 10;
the nucleotide sequence of the CD3ξ domain is SEQ ID NO: 11;
the amino acid sequence of the CD3ξ domain is SEQ ID NO: 12.

Preferably, in S1, steps for constructing the recombinant lentiviral plasmid pCDH-EF1α-B7H3-CAR include:
S1-1, performing a double digestion for the lentiviral backbone plasmid pCDH-EF1α with BamHI and NotI restriction enzymes, and recovering a product by gel extraction;
S1-2, mixing a fragment recovered from S1-1 and the fragment containing CD8α leader, B7H3 scFV, CD8 hinge region, CD28 transmembrane domain, 4-1BB intracellular domain and CD3ξ domain at a molar ratio of 1:1, linking with a T4 DNA ligase, and then transforming into a Stbl3 competent cell;
S1-3, selecting a monoclonal cell of the Stbl3 competent cell for plasmid extraction, and the plasmid is pCDH-EF1α-B7H3-CAR.

Preferably, in S2, the specific steps for linking artificial miRNA fragments are as follows:
S2-1, performing a double digestion for the plasmid pCDH-EF1α-B7H3-CAR obtained in S1 with BclI and HpaI for gel extraction;
S2-2, performing a double digestion for the artificial miRNA fragments with BclI and HpaI for gel extraction;
S2-3, linking the fragment recovered in S2-1 with the fragment recovered from step S2-2 by the T4 DNA ligase, and then transforming into the Stb13 competent cell;
S2-4, selecting the monoclonal cells of Stb13 competent cells for plasmid extraction, and the plasmid is pCDH-EF1α-miRNA-B7H3-CAR.

Preferably, in S4, activating and amplifying a human T-cell for 5 days, and re-infecting the obtained T-cell with pCDH-EF1α-miRNA-B7H3-CAR lentivirus.

A method for preparing enhanced CAR-T based on knocking out the human PCSK9 gene, the method is performed by using the above ready-to-use sgRNA, including the following steps:
(1) cloning CD8α leader, B7H3 scFV, CD8 hinge region, CD28 transmembrane domain, 4-1BB intracellular domain and CD3& domain into the lentiviral backbone plasmid pCDH-EF1α to construct the recombinant lentiviral plasmid pCDH-EF1α-B7H3-CAR;
(2) co-transfecting the recombinant lentiviral plasmid pCDH-EF1α-B7H3-CAR and the helper plasmid pMD2.G/pSPAX2 obtained in Step (1) into 293T-cells for packaging to obtain pCDH-EF1α-B7H3-CAR lentivirus;
(3) using the pCDH-EF1α-B7H3-CAR lentivirus obtained in Step (2) to activate and amplify T-cells cultured for 5 days in vitro to obtain a B7H3-CAR-T-cell;
(4) by CRISPR-Cas9 technology, transferring a ribonucleoprotein RNP formed by the ready-to-use sgRNA and Cas9 protein into the B7H3-CAR-T-cell obtained in Step (3), and specifically targeting and knocking out the human PCSK9 gene to obtain a sgPsck9-B7H3-CAR-T-cell.

Preferably, in Step (4), the gene knocking out process includes delivering the RNP complex into the B7H3-CAR-T-cell by electroporation for gene editing to prepare the sgPsck9-B7H3-CAR-T-cell.

Therefore, the invention adopts the above method for preparing enhanced CAR-T-cells based on interfering or knocking out the human PCSK9 gene and the application thereof, which has the following technical effects:
(1) The invention uses artificial miRNA to overcome the weakness of previous RNA interference technology that is easy to miss the target, and can efficiently and specifically silence the target gene, in addition, the invention integrates miRNA into the CAR expression vector, which can simultaneously interfere with the target gene and CAR expression;
(2) In this invention, the purified Cas9 protein and guide RNA are used to form RNP (RNA-protein complex) in vitro, and then introduced into CAR-T cells for gene editing using chemical transfection or electroporation, direct delivery of Cas9/sgRNA RNP does not require transcription and translation, and can achieve rapid gene editing, at the same time, it has a shorter half-life, a lower off-target rate, and is more safe and effective.
(3) Specific knockdown or knockout of PCSK9 expression in CAR-T-cells by PCSK9 gene knockdown element or CRISPR/Cas9 technology can effectively enhance the anti-tumor effect of CAR-T-cells and the tumor treatment effect of immune checkpoint therapy.

### BRIEF DESCRIPTION OF THE DRAWINGS

FIGS. 1A-1B are schematic diagrams of interfering or knocking out the human PCSK9 gene, where FIG. 1A is a schematic diagram of vector elements containing gene knockdown miRNA and CAR, FIG. 1B is a schematic diagram of vector elements expressing CAR and gene knockout by electroporation in CAR-T-cells;

FIG. 2 is a CAR positive rate of PBMC cells, miR-scr-B7H3-CAR-T-cells, miR-603-B7H3-CAR-T-cells, miR-792-B7H3-CAR-T-cells, sg-scr-B7H3-CAR-T-cells, sg #1-B7H3-CAR-T-cells and sg #6-B7H3-CAR-T-cells.

FIGS. 3A-3B are detection results of PCSK9 interference efficiency, where FIG. 3A is an expression of PCSK9 in miR-scr-B7H3-CAR-T-cells, miR-#603-B7H3-CAR-T-cells and miR-#792-B7H3-CAR-T-cells; FIG. 3B is an expression of PCSK9 in sg-scr-B7H3-CAR-T-cells, sg #1-B7H3-CAR-T-cells and sg #6-B7H3-CAR-T-cells;

FIGS. 4A-4B show in vitro anti-tumor activity of CAR-T-cells; among them, FIG. 4A is the killing statistics of miR-scr-B7H3-CAR-T-cells, miR-#603-B7H3-CAR-T-cells and miR-#792-B7H3-CAR-T-cells on pancreatic cancer cells. FIG. 4B is the killing statistics of sg-scr-B7H3-CAR-T-cells, sg #1-B7H3-CAR-T-cells and sg #6-B7H3-CAR-T-cells on pancreatic cancer cells;

FIG. 5 is an in vivo imaging of small animals of tumor-bearing mice on the 10th, 15th, and 20th day of CAR-T-cells anti-tumor activity in vivo;

FIG. 6 is a tumor size of each group of tumor-bearing mice in the detection of anti-tumor activity of CAR-T-cells in vivo;

FIG. 7 is the tumor weight statistics of each group of tumor-bearing mice in the anti-tumor activity detection of CAR-T-cells in vivo.

### DETAILED DESCRIPTION OF THE EMBODIMENTS

The following is a further explanation of the technical scheme of the invention through drawings and embodiments.

Unless otherwise defined, the technical terms or scientific terms used in the invention should be understood by people with general skills in the field to which the invention belongs.

### Embodiment 1

A method for preparing enhanced CAR-T-cells based on interfering or knocking out the human PCSK9 gene includes the following steps:

### S1, Construction of Lentiviral Expression Vector

Expression vector 1: pCDH-EF1α-B7H3-CAR lentiviral expression vector, the sequence includes CD8α leader, B7H3 scFV, CD8 hinge region, CD28 transmembrane domain, 4-1BB intracellular domain, and CD3ξ domain.

Expression vector 2: pCDH-EF1α-miRNA-B7H3-CAR lentiviral expression vector, the sequence includes miRNA sequence inserted into EF1α intron region, CD8α leader, B7H3 scFV, CD8 hinge region, CD28 transmembrane domain, 4-1BB intracellular domain and CD3ξ domain.

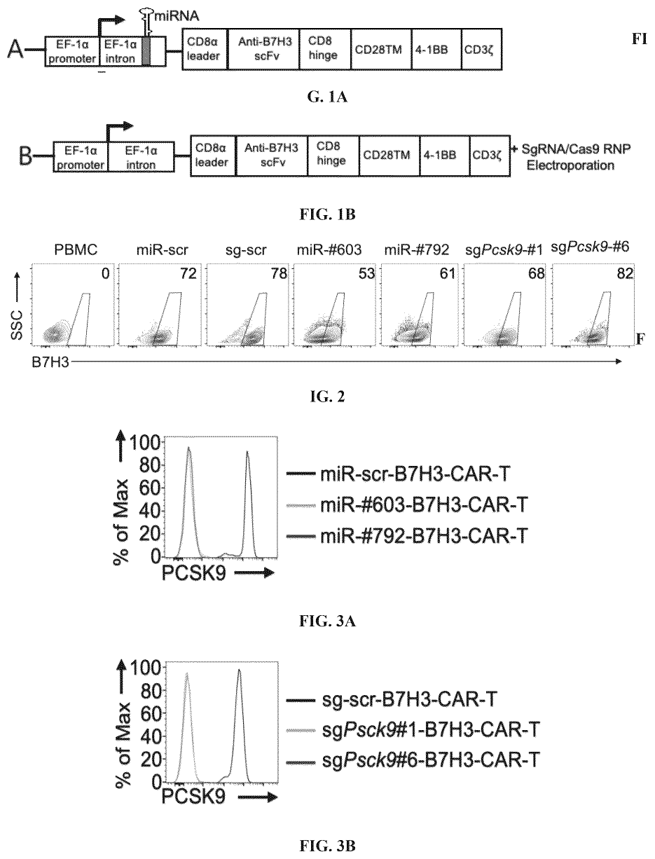

The domain sequence of CD8α leader, B7H3 scFV, CD8 hinge region, CD28 transmembrane domain, 4-1BB intracellular domain, and CD3ξ domain is commissioned by GenScript Biotechnology Corporation (Nanjing) to synthesize the entire expression cassette, as shown in FIGS. 1A-1B.

The nucleotide sequence of the CD8α leader is
atggccttaccagtgaccgccttgctcctgccgctggccttgctgctcca
cgccgccaggccg, as shown in SEQ ID NO: 1
the amino acid sequence of the CD8α leader is
MALPVTALLLPLALLLHAARP, as shown in SEQ ID NO: 2;
the nucleotide sequence of the B7H3 scFV is
ctgcatgtaggctgtgctggaggattcgtctacagtcaatgtgaccttgt
ccttgaactcttgattgtagttagtataaatataagaaggataaatattt
ccgatccactcaaggccttgtccaggcctctgcttcacccagtttatcca
gtagttggtgaaggtgtagccagaagccttgcaggacagcttcacagccc
caggcctcaccagttcagccccctgctgcagcttgacctgctcagcagcc
cgacatctgaggactctgcggtctattactgtacaagatccccttatggt
tacgacgagtatggtctggactactggggccaaggcaccacggtcaccgt
ctcctcaggtggTggcggttcaggcggaggtggctctggcggtggcggat
cggacatcgagctcactcagtctccatcctccctgactgtgacagcagga
gagaaggtcactatgaactgcaagtccagtcagagtctgttaaacagtag
aaatcaaaagaactacttgacctggtaccagcagaaaccagggcagcctc
ctaaactgttgatatactgggcatccactagggaatctggggtccctgat
cgcttcacaggcagtggatctggaacagatttcactctcaccatcagcag
tgtgcaggctgaagacctggcagtttattactgtcagaatgattatgttt
atccgctcacgttcggtgctGggaccaagctggaaataaaacgg,
as shown in SEQ ID NO: 3;
the amino acid sequence of the B7H3 scFV is
LHVGCAGGFVYSQCDLVLELLIVVSINIRRINISDPLKALSRPLLHPVYP
VVGEGVARSLAGQLHSPRPHQFSPLLQLDLLSSPTSEDSAVYYCTRSPYG
YDEYGLDYWGQGTTVTVSSGGGGSGGGGSGGGGSDIELTQSPSSLTVTAG
EKVTMNCKSSQSLLNSRNQKNYLTWYQQKPGQPPKLLIYWASTRESGVPD
RFTGSGSGTDFTLTISSVQAEDLAVYYCONDYVYPLTFGAGTKLEIKR,
as shown in SEQ ID NO: 4;
the amino acid sequence of the CD8 hinge region is
accacgacgccagcgccgcgaccaccaacaccggcgcccaccatcgcgtc
gcagcccctgtccctgcgcccagaggcgtgccggccagcggcggggggcg
cagtgcacacgagggggctggacttcgcctgtgat, as shown in
SEQ ID NO: 5;
the amino acid sequence of the CD8 hinge region is
TTTPAPRPPTPAPTIASQPLSLRPEACRPAAGGAVHTRGLDFACD, as
shown in SEQ ID NO: 6;
the nucleotide sequence of the CD28 transmembrane
domain is
ttttgggtgctggtggtggttggtggagtcctggcttgctatagcttgct
agtaacagtggcctttattattttctgggtgaggagtaagaggagcaggc
tcctgcacagtgactacatgaacatgactccccgccgccccgggcccacc
cgcaagcattaccagccctatgccccaccacgcgacttcgcagcctatcg
ctcc, as shown in SEQ ID NO: 7;
the amino acid sequence of the CD28 transmembrane
domain is
FWVLVVVGGVLACYSLLVTVAFIIFWVRSKRSRLLHSDYMNMTPRRPGPT
RKHYQPYAPPRDFAAYRS, as shown in SEQ ID NO: 8;
the nucleotide sequence of the 4-1BB intracellular
domain is
aaacggggcagaaagaaactcctgtatatattcaaacaaccatttatgag
accagtacaaactactcaagaggaagatggctgtagctgccgatttccag
aagaagaagaaggaggatgtgaactg, as shown in SEQ ID NO:
9;
the amino acid sequence of the 4-1BB intracellular
domain is
KRGRKKLLYIFKQPFMRPVQTTQEEDGCSCRFPEEEEGGCEL, As
shown in SEQ ID NO: 10;
the nucleotide sequence of the CD3ξ domain is
agagtgaagttcagcaggagcgcagacgcccccgcgtaccagcagggcca
gaaccagctctataacgagctcaatctaggacgaagagaggagtacgatg
ttttggacaagagacgtggccgggaccctgagatggggggaaagccgaga
aggaagaaccctcaggaaggcctgtacaatgaactgcagaaagataagat
ggcggaggcctacagtgagattgggatgaaaggcgagcgccggaggggca
aggggcacgatggcctttaccagggtctcagtacagccaccaaggacacc
tacgacgcccttcacatgcaggccctgccccctcgc, as shown in
SEQ ID NO: 11;
the amino acid sequence of the CD3ξ domain is
RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR, as shown in SEQ ID NO: 12.

The fusion gene fragment of the CD8α leader, B7H3 scFV, CD8 hinge region, CD28 transmembrane domain, 4-1BB intracellular domain, and CD3ξ domain is cloned into the lentiviral backbone plasmid pCDH-EF1α to obtain the recombinant lentiviral plasmid pCDH-EF1α-B7H3-CAR.

The miRNA fragment is further linked into the EF1α intron region of the pCDH-EF1α-B7H3-CAR plasmid to obtain the recombinant lentiviral plasmid pCDH-EF1α-miRNA-B7H3-CAR after PCSK9 knocking down.

The specific steps include:

S1-3. The single clone was selected for plasmid extraction. After enzyme digestion and sequencing, the plasmid was confirmed to be pCDH-EF1α-B7H3-CAR.

S1-4, the synthesized miRNA #603 with miR-155 as the backbone was double digested with BclI and HpaI for gel extraction.

S1-1, the double digestion is performed for the lentiviral backbone plasmid pCDH-EF1α with BamHI and NotI restriction enzymes, and the product is recovered by gel extraction.

S1-2, the fragment recovered from S1 is synthesized with the sequence of the entire expression cassette of the CD8α leader, B7H3 scFV, CD8 hinge region, CD28 transmembrane domain, 4-1BB intracellular domain and CD3ξ domain by GenScript Biotechnology Corporation (Nanjing), it is mixed according to the molar ratio of 1:1, the T4 enzyme is used for linking, and then it is transformed into the Stb13 competent cells.

S1-3, the single clone is selected for plasmid extraction, after enzyme digestion identification, it is sent for sequencing confirmation, and the plasmid is confirmed to be pCDH-EF1α-B7H3-CAR.

S1-4, the synthesized miRNA #603 with miR-155 as the backbone is double digested with BclI and HpaI for gel extraction.

Where the miRNA sequence in the stem-loop structure is replaced by the sequence of miRNA #603-guide sequence/miRNA #603-passenger sequence with miR-155 as the backbone, the restriction enzyme sites of Bcl1 and Srf1 are added to the 5′ end, and the restriction enzyme sites of HpaI and Sph1 are added to the 3′ end for chemical synthesis.

The full-length sequence corresponding to miRNA #603, as shown in SEQ ID NO: 19, is tggaggcttgctttgggctgtatgctgacattctcgaagtcggtgaccgttttggccactgactgacggtcacgactcgagaatgtcaggaca caaggccctttatcagcactcacatggaacaaatggcccgttaacgta.

The nucleotide sequence of miRNA #603-guide sequence is acattctcgaagtcggtgacc, as shown in SEQ ID NO: 13;

the nucleotide sequence of miRNA #603-passenger sequence is ggtcacgactcgagaatgt, as shown in SEQ ID NO: 14.

S1-5, the product obtained in S1-3 is linked with the product obtained in S1-4 by the T4 enzyme, and then transformed into the Stbl3 competent cells.

S1-6, single clones are selected for plasmid extraction, after enzyme digestion identification, it is sent for sequencing confirmation, the plasmid is pCDH-EF1α-miRNA-#603-B7H3-CAR.

### S2, Lentivirus Packaging and Preparation

S2-1, Day 1: 5×10⁶ 293T-cells were centrifuged and the supernatant was discarded, the cells were resuspended in 10 mL complete medium (D-MEM+10% FBS+2 mML-glutamine+0.1 mM non-essential amino acids+1 mM sodium pyruvate+1% P/S) preheated at 37° C. and inoculated in a 10 cm culture plate, and it was incubated overnight at 37° C. in a 5% CO₂ incubator.

S2-2, the culture medium in the culture plate was discarded, and 5 mL of Opti-MEM® I culture medium containing 10% FBS was added.

S2-3, Preparation of DNA-Lipofectamine® 3000 complex:

S2-3-1. 1.5 mL of serum-free Opti-MEM® I culture medium was added to 5 mL centrifuge tube, the 9 μgpMD2.G and pSPAX2 packaging plasmid mixture and 3 μgpCDH-EF1α-B7H3-CAR or pCDH-EF1α-miRNA #603-B7H3-CAR were added and gently mixed.

S2-3-2, 1.5 mL of serum-free Opti-MEM® I medium was added to another 5 mL centrifuge tube, and 36 μL of Lipofectamine® 3000 was added, gently mixed, and incubated at room temperature for 5 min.

S2-3-3, the solution obtained by S2-3-1 and S2-3-2 was transferred to a centrifuge tube and gently mixed evenly.

S2-3-4, the mixture was incubated at room temperature for 20 min to obtain the DNA-Lipofectamine® 3000 complex.

S2-4, the obtained DNA-Lipofectamine® 3000 complex was slowly added to the culture plate drop by drop, and gently swirled the culture plate to ensure homogeneous distribution of complex to the entire well. It was incubated overnight at 37° C. in the 5% CO₂ incubator.

S2-5, Day 3: The culture plate was taken out, carefully aspirated the media, the media was replaced with 10 mL of DMEM complete medium. It was incubated overnight at 37° C. in a 5% CO₂ incubator for 72 h.

S2-6, Day 5 or Day 6: The culture medium in the culture plate was transferred to a 15 mL centrifuge tube and centrifuged at 2000 g at 4° C. for 15 min.

S2-7, The 0.45 μm filter was used to filter the supernatant into 50 mL 100 kDa ultrafiltration tubes, with 12 mL in each tube. Subsequently, the tubes were centrifuged at 5000 g for 30 minutes at 4° C.

S2-9, the virus concentrate was obtained and aliquoted into a sterilized EP tube and stored at −80° C.

S3, preparation of B7H3-CAR-T-cells, B7H3-CAR-T-cells with PCSK9 knockdown or knockout.

$3-1, preparation of culture medium: Preparation of a 50 mL aliquot of complete medium. The complete medium contains 1.5 mL of CTS Immune Cell SR+0.5 mL of GlutaMax+1.3 mL of CTS OpTmizer T-Cell Expansion Basal Medium Supplement+0.5 mL of P/S+25 μL of IL-2 (100 IU), CTS OpTmizer T-Cell Expansion Basal Medium was added to make up the volume to 50 mL.

S3-2, PBMC isolation

S3-2-1, the human peripheral blood mononuclear cell separation solution was restored to room temperature 30 min in advance.

S3-2-2, the fresh heparin-anticoagulated peripheral blood from patients was gently mixed with FACS (PBS+2% FBS) in a 1:1 ratio until uniform.

S3-2-3, a new sterile centrifuge tube was taken and the cell separation liquid was placed at the bottom of the tube, the diluted peripheral blood and the separation liquid with the same volume as the lymphocyte separation liquid were gently spread along the wall of the tube on the upper layer of the lymphocyte separation liquid.

S3-2-4, it was centrifuged at 800 g at room temperature for 25 min with brake off. The centrifugal tube was divided into four layers from top to bottom. The first layer was the DPBS layer, the second layer was mononuclear cells, the third layer was the transparent separation liquid layer, and the fourth layer was the red blood cell layer.

S3-2-5, the cells in the mononuclear cells layer were carefully drawn into a new 50 mL centrifuge tube, RPMI1640 was added until the liquid reached 50 mL, and then it was centrifuged at 600 g for 10 min, the supernatant was discarded; the cells were re-suspended with 50 mL RPMI1640 and centrifuged at 500 g for 10 min, and the supernatant was discarded. The precipitate consists of PBMC.

S3-3, T-cell culture, virus infection, and electroporation.

Day 0: The complete medium prepared in Step S3-1 was aspirated, and the PBMC cells obtained in Step 2-5 were re-suspended at a concentration of 1.5×10⁶ cells/mL. CD3/28 magnetic beads were added at a 1:1 ratio. The mixture was placed in a incubator (37° C., 5.0% CO₂, saturated humidity) for culture.

Day 1: No treatment.

Day 2: No treatment.

Day 3: The cells in the orifice plate were gently blown up and collected into a 50 ml centrifuge tube and centrifuged at 1000 rpm for 5 min, and the supernatant was discarded. After resuspending in 5 mL medium, the medium was transferred to a sterile flow tube and placed on a magnet (Stemcell), stood for 2 min, and the supernatant was collected (the purpose of this step is to remove the magnetic beads). The cells were counted and the cell density was adjusted to 1.5×10⁶/mL, the cells were transferred to a culture flask or petri dishes to continue the culture.

Day 4: The undisturbed was left without any treatment.

Day 5: After activation and amplification, T-cells were obtained. The cells were collected (5×10⁶ cells), re-suspended in 500 μL fresh medium, then transferred into a new 48-orifice plate. Each well was added with 100 μL of concentrated lentivirus (evenly pipetted into the wells). The plate was then covered, mixed well, and incubated in an incubator for 4 hours before adding 4.5 mL of medium. The cells were then cultured overnight. Day 6: Follow the steps of Day 5 for secondary infection.

Day 7: The T-cells that had been infected twice were changed and the density of T-cells was adjusted to 1.5×10⁶/mL. B7H3-CAR-T-cells and miR-603-B7H3-CAR-T-cells were obtained by this step, and the structure diagram is shown in FIGS. 1A-1B.

Day 8: The undisturbed was left without any treatment.

Day 9: Electroporation was conducted.
1) B7H3-CAR-T-cells that need electroporation were collected, and gently pipette to ensure thorough collection of the cells from the bottom of culture plate. Centrifugation was performed at 300 g for 5 min and the supernatant was discarded. The cells were counted for later use.
2) Gene editing preparation: A sterile EP tube was taken and 20 μL of R buffer was added. 3 μg Cas9 protein per 10⁶ cells (or 18 pmol/10⁶ cells) and 45 pmol sgRNA #6 per 10⁶ cells were added, the mixture was incubated for 15 min at room temperature. The Cas9 protein and sgRNA can be adjusted according to the actual situation (optimized according to efficiency).

The sequence of sgRNA #6spacer is cuuggcaguugagcacgcgc, as shown in SEQ ID NO: 23; chemically synthesized ready-to-use sgRNA, the nucleotide sequence of the ready-to-use sgRNA #6 is cuuggcaguugagcacgcgcguuuuagagcuagaaauagcaaguuaaaauaaggcuaguccguuaucaacuugaaaaagugg caccgagucggugcuuuu, as shown in SEQ ID NO: 25.
3) During the incubation of Cas9 protein and sgRNA #6, 1.5×10⁶ B7H3-CAR-T-cells were taken, after centrifugation at 300 g for 5 min, the supernatant was completely removed (washed again with DPBS without calcium and magnesium), and 1.5×10⁶ cells were resuspended with 80 μL R buffer.
4) During the incubation of Cas9 and sgRNA #6, the Neon Transfection instrument was assembled, and 3 mL of E buffer was added to the electric tube.
5) During the incubation of Cas9 and sgRNA #6, 2.5 mL of T-cell complete medium (supplemented with 10% FBS) was taken into a 6-orifice plate, and the medium was placed in the incubator for rewarming.
6) The cells resuspended in R buffer were transferred to Cas9 and sgRNA #6 incubation tubes, and the cells were mixed well, the total volume was prepared to about 110 μL, and R buffer could be added if it was insufficient.
7) The electronic pipette was used to aspirate 100 μL of the cell mixture, pipette projection was inserted into the groove of pipette station.
8) The electroporation program 1700/20/1 was selected on the Neon electroporation instrument.
9) After the electroporation was completed, the pipette was removed slowly from the pipette ptation and the samples was transferred immediately from the tip by pressing the plunger on the pipette to the first stop into the prepared culture plate containing prewarmed medium (culture density is 2×10⁶/mL).
10) After 24 hours of gene editing, if the medium becomes yellow, the T-cell complete medium can be used to adjust the cell culture density to 1×10⁶/mL. This step obtained the PCSK9 knockout sg #6-B7H3-CAR-T-cells.
11) After 72 hours of gene editing, the cell phenotype and gene editing efficiency could be sampled and detected.

### Embodiment 2

A method for preparing enhanced CAR-T based on interfering or knocking out the human PCSK9 gene adopts the same method as Embodiment 1, the difference is that:

The miRNA fragment connected to pCDH-EF1α-B7H3-CAR was miRNA #792, and pCDH-EF1α-miRNA #792-B7H3-CAR was prepared.

The PCSK9 knockdown cell was miR-#792-B7H3-CAR-T;

the sgRNA during electroporation was sgRNA #1, and the prepared PCSK9 knockout cell was sg #1-B7H3-CAR-T.

The miRNA sequence in the stem-loop structure with miR-155 as the backbone was replaced by the sequence of the miRNA #792-guide sequence/passenger sequence, the Bell and Srf1 restriction sites were added at the 5′ end, and the Hpa13 and Sph1 restriction sites were added at the 3′ end for chemical synthesis.

miRNA#792-guide sequence: ataaactccaggcctatgagg,
as shown in SEQ ID NO: 15.
miRNA#792-passenger sequence: cctcatggctggagtttat,
as shown in SEQ ID NO: 16.

The corresponding full-length sequence of miRNA #792 is tggaggcttgctttgggctgtatgctgataaactccaggcctatgagggttttggccactgactgaccctcatggctggagtttatcaggacac aaggccctttatcagcactcacatggaacaaatggcccgttaacgtagcatgcatgta, as shown in SEQ ID NO: 20.

The nucleotide sequence of sgRNA #1 spacer is uacaggcagcaccagcgaag, as shown in SEQ ID NO: 22; the chemical synthesis of ready-to-use sgRNA, the nucleotide sequence of the ready-to-use is sgRNA #1 uacaggcagcaccagcgaagguuuuagagcuagaaauagcaaguuaaaauaaggcuaguccguuaucaacuugaaaaaguggc accgagucggugcuuuu, as shown in SEQ ID NO: 24.

### Embodiment 3

A method for preparing enhanced CAR-T based on interfering or knocking out the human PCSK9 gene adopts the same method as Embodiment 1. The difference is that:

The miRNA fragment connected to pCDH-EF1α-B7H3-CAR was miR-scramble, and pCDH-EF1α-miRNA-scramble-B7H3-CAR was prepared; PBMC cells were infected with pCDH-EF1α-miRNA-scramble-B7H3-CAR lentivirus to obtain miR-scr-B7H3-CAR-T;

the sgRNA during electroporation was sg-scramble, and the prepared cell was sg-scr-B7H3-CAR-T.

The miRNA sequence in the stem-loop structure with miR-155 as the backbone was replaced by the sequence of the miR-scramble-guide sequence/passenger sequence. The Bcl1 and Srf1 restriction sites were added at the 5′ end, and the Hpa13 and Sph1 restriction sites were added at the 3′ end for chemical synthesis.

miR-scramble-guide sequence: ttctaatactacgttccgcat, as shown in SEQ ID NO: 17.

miR-scramble-passenger sequence: atgeggacgagtattagaa, as shown in SEQ ID NO: 18.

The full-length sequence corresponding to miR-scramble is tggaggcttgctttgggctgtatgctgttctaatactacgttccgcatggttttggccactgactgacatgcggacgagtattagaacaggaca caaggccctttatcagcactcacatggaacaaatggcccgttaacgta, the nucleotide sequence is shown in SEQ ID NO: 21.

The nucleotide sequence of sg-scramble is ggccgaaaccugauccuuuaguuuuagagcuagaaauagcaaguuaaaauaaggcuaguccguuaucaacuugaaaaagugg caccgagucggugcuuuu, as shown in SEQ ID NO: 26.

### Experimental Test

### (1) Detection of CAR Expression Efficiency.

The positive rates of PBMC cells, miR-scr-B7H3-CAR-T-cells, miR-#603-B7H3-CAR-T-cells, miR-#792-B7H3-CAR-T-cells, sg-scr-B7H3-CAR-T-cells, sgPcsk #1-B7H3-CAR-T-cells and sgPcsk #6-B7H3-CAR-T-cells were detected by flow cytometry. CAR-T-cells were co-incubated with B7H3 antigen protein at room temperature for 60 min and then washed. Then it was co-incubated with an anti-B7H3 antibody, after the incubation at 4° C. for 30 min, after the cells were washed, the cells were analyzed by flow cytometry.

The results are shown in FIG. 2, PBMC cells were negative control group. The results showed that both miR-B7H3-CAR-T-cells and sg-B7H3-CAR-T-cells could successfully express CAR that recognizes B7H3 (the data in the figure are expressed in %).

### (2) Detection of PCSK9 Interference Efficiency

The expression of PCSK9 in miR-scr-B7H3-CAR-T-cells, miR-#603-B7H3-CAR-T-cells, miR-#792-B7H3-CAR-T-cells, sg-scr-B7H3-CAR-T-cells, sg #1-B7H3-CAR-T-cells and sg #6-B7H3-CAR-T-cells were detected by flow cytometry.

After the cell membranes were broken, the cells were washed and incubated with anti-PCSK9 antibody. After incubation at 4° C. for 30 min, the cells were washed and the flow cytometry was used for analysis.

As shown in FIGS. 3A-3B, the expressions of PCSK9 were significantly decreased in miR-#603-B7H3-CAR-T-cells, miR-#792-B7H3-CAR-T-cells, sgPcsk #1-B7H3-CAR-T-cells, and sgPcsk #6-B7H3-CAR-T-cells.

### (3) Detection of the Anti-Tumor Activity of CAR-T-Cells In Vitro.

(1) The miR-B7H3-CAR-T and sg-B7H3-CAR-T were used as effector cells, and Panc01-luc pancreatic cancer cells were used as target cells. The effector cells and target cells were mixed in 96-orifice plates according to the effector-target ratio of 1:1, 5:1, 10:1, and gently mixed; at the same time, a cell-free culture medium hole and a sample control hole with only target cells were set up and incubated at 37° C. in a 5% CO₂ incubator.
(2) At 18 h after co-incubation, the lysis buffer and luciferase substrate were added and the fluorescence intensity was detected using a microplate reader.

$$Cytotoxicity / mortality = ( absorbance of treated sample - absorbance of sample control hole ) / absorbance of sample control hole \times 100 \% .$$

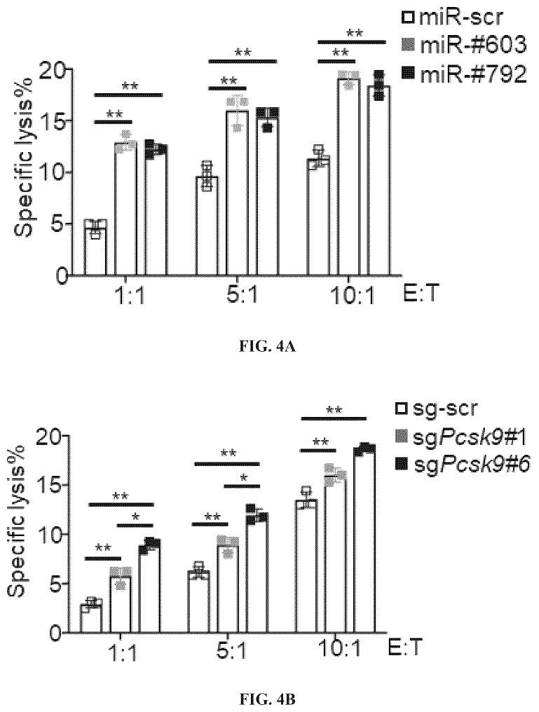

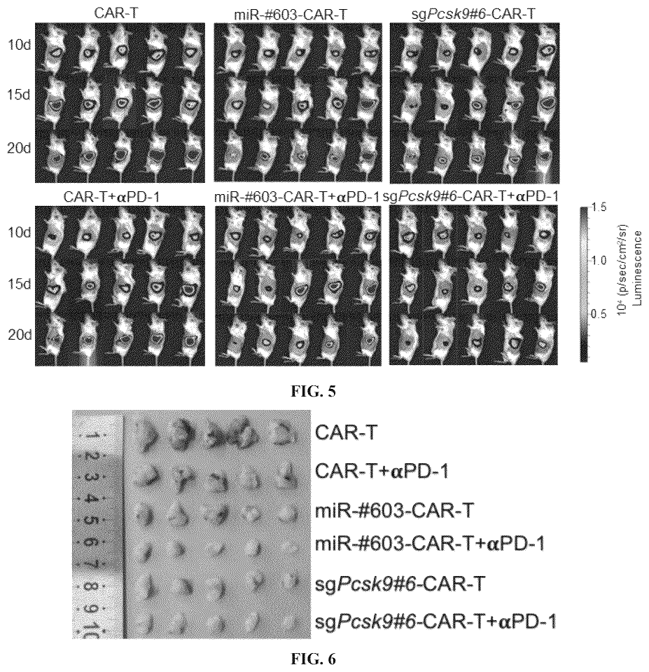

The experimental results are shown in FIGS. 4A-4B. FIG. 4A is the killing statistics of miR-scr-B7H3-CAR-T-cells, miR-#603-B7H3-CAR-T-cells, and miR-#792-B7H3-CAR-T-cells on pancreatic cancer cells, FIG. 4B is the killing statistics of sg-scr-B7H3-CAR-T-cells, sg #1-B7H3-CAR-T-cells, and sg #6-B7H3-CAR-T-cells on pancreatic cancer cells.

The results showed that the killing rates of miR-#603-B7H3-CAR-T-cells, miR-#792-B7H3-CAR-T-cells, sg #1-B7H3-CAR-T-cells and sg #6-B7H3-CAR-T-cells on Pan01-luc pancreatic cancer cells were significantly stronger than those of ordinary miR-scr-B7H3-CAR-T-cells and sg-scr-B7H3-CAR-T-cells. It was indicated that the PCSK9 knockdown or knockout CAR-T-cells prepared by the invention have an efficient killing effect on pancreatic cancer cells.

### (4) Detection of Anti-Tumor Activity of CAR-T-Cells In Vivo

Panc01-luc human pancreatic cancer cells in the logarithmic growth phase were digested by trypsin and made into a single-cell suspension. 0.1 mL of suspension containing 1× 10⁶ cancer cells was in situ inoculated into the pancreatic tissue of NCG mice.

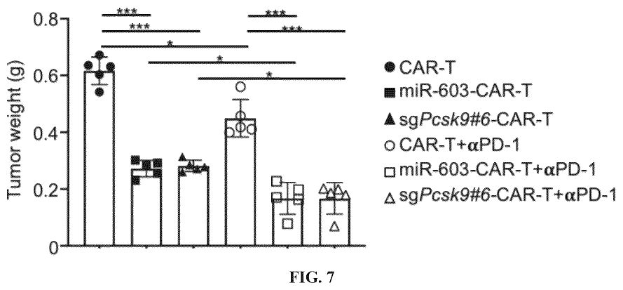

The experimental grouping and treatment are shown in Table 1, the changes in diet and activity of each group of animals were observed daily, and the body weight of mice was measured every other day to observe the change in body weight. On the 10th, 15th, and 20th day after tumor-bearing, the growth of the tumor, experimental grouping, and treatment were monitored by the small animal in vivo imaging system. On the 10th, the 15th, and the 20th day, the small animal in vivo imaging of tumor-bearing mice is shown in FIG. 5, and the tumor size of tumor-bearing mice in each group is shown in FIG. 6.

| Group | Treatment | Note |
|---|---|---|
| (1) 5 mice in the control | 5 × 10⁶ B7H3-CAR-T was | After treatment, the animals |
| group | injected into the tail veins | in each group were observed |
| | of mice, and the infusion was | for obvious abnormal |
| | repeated every 5 days for 3 | reactions. |
| | times | (Toxic reaction, allergic |
| (2) 5 mice in the miRNA- | 5 × 10⁶ miR-603-B7H3-CAR-T | reaction, local stimulation |
| B7H3-CAR-T treatment | was injected into the tail veins of | reaction, etc.). |
| group | mice, and the infusion was repeated | |
| | every 5 days for 3 times | |
| (3) 5 mice in the sg- | 5 × 10⁶ sg#6-B7H3-CAR-T | |
| B7H3-CAR-T treatment | was injected into the tail veins | |
| group | of mice, and the infusion was | |
| | repeated every 5 days for 3 | |
| | times | |
| (4)5 mice in the miR-B7H3- | 5 × 10⁶ miR-#603-B7H3- | |
| CAR-T combined with | CAR-T was injected into the | |
| PD-1 treatment group | tail veins of mice, and the | |
| | infusion was repeated every 5 | |
| | days, PD-1 was given on the | |
| | second day of CAR-T cell | |
| | therapy, and the treatment is | |
| | last for 3 times | |
| (5)5 mice in the sg-B7H3- | 5 × 10⁶ sg#6-B7H3-CAR-T | |
| CAR-T combined with PD- | was injected into the tail veins | |
| 1 treatment group | of mice, and the infusion was | |
| | repeated every 5 days, PD-1 | |
| | was given on the second day of | |
| | CAR-T cell therapy, and the | |
| | treatment is last for 3 times | |

It was found that the growth of tumors in mice treated with PCSK9 knockout or knockdown B7H3-CAR-T was inhibited to a certain extent, and the therapeutic effect of PD-1 was significantly improved. This suggests that the CAR-T constructed in this patent, which interferes with PCSK9, can markedly suppress tumor growth in vivo and improve the effectiveness of immunotherapy.

Finally, it should be explained that the above embodiments are only used to explain the technical scheme of the invention rather than restrict it. Although the invention is described in detail concerning the better embodiments, the ordinary technical personnel in this field should understand that they can still modify or replace the technical scheme of the invention, and these modifications or equivalent substitutions cannot make the modified technical scheme out of the spirit and scope of the technical scheme of the invention.

What is claimed is:

1. A method for preparing a chimeric antigen receptor T-cell (CAR-T-cell), comprising administering an artificial miRNA or an sgRNA to knock out the human PCSK9 gene and prepare the CAR-T-cell;
wherein the artificial miRNA is prepared by using miR-155 as a skeleton and replacing a miRNA sequence in a stem-loop structure of the miR-155 with a miRNA fragment, wherein the miRNA fragment targets expression of the human PCSK9 gene,
wherein the miRNA fragment comprises a miRNA #603-guide sequence, a miRNA #603-passenger sequence, a miRNA #792-guide sequence, or a miRNA #792-passenger sequence;
wherein the nucleotide sequence of the miRNA #603-guide sequence comprises SEQ ID NO: 13,
the nucleotide sequence of the miRNA #603-passenger sequence comprises SEQ ID NO: 14;
the nucleotide sequence of the miRNA #792-guide sequence comprises SEQ ID NO: 15,
the nucleotide sequence of the miRNA #792-passenger sequence comprises SEQ ID NO: 16;
wherein the sgRNA comprises sgRNA #1 or sgRNA #6, wherein the spacer sequence of the sgRNA #1 is SEQ ID NO: 22, and the spacer sequence of the sgRNA #6 is SEQ ID NO: 23;
wherein, after the sgRNA is chemically synthesized into a ready-to-use sgRNA, the ready-to-use sgRNA is applied to knock out the human PCSK9 gene to prepare the CAR-T-cell;
wherein the ready-to-use sgRNA comprises ready-to-use sgRNA #1 or ready-to-use sgRNA #6, wherein the nucleotide sequence of the ready-to-use sgRNA #1 comprises SEQ ID NO: 24, and the nucleotide sequence of the ready-to-use sgRNA #6 comprises SEQ ID NO: 25.

2. The method for preparing a CAR-T-cell according to claim 1, wherein the method comprises the following steps:
Step 1, cloning a fusion gene fragment encoding a CD8α leader, a B7H3 scFV, a CD8 hinge region, a CD28 transmembrane domain, a 4-1BB intracellular domain, and a CD3ξ domain into a lentiviral backbone plasmid pCDH-EF1α to construct a recombinant lentiviral plasmid pCDH-EF1α-B7H3-CAR;
Step 2, linking the artificial miRNA into the recombinant lentiviral plasmid pCDH-EF1α-B7H3-CAR constructed in the S1 to obtain a PCSK9 knockdown recombinant lentiviral plasmid pCDH-EF1α-miRNA-B7H3-CAR;
Step 3, co-transfecting the pCDH-EF1α-miRNA-B7H3-CAR and a helper plasmid pMD2.G/pSPAX2 into 293T-cells for packaging to obtain a pCDH-EF1α-miRNA-B7H3-CAR lentivirus; and
Step 4, using the pCDH-EF1α-miRNA-B7H3-CAR lentivirus obtained in the S3 to infect an activated T-cell in vitro to obtain a PCSK9 knockdown miR-B7H3-CAR-T-cell;
wherein the nucleotide sequence of the CD8α leader is SEQ ID NO: 1;
the amino acid sequence of the CD8α leader is SEQ ID NO: 2;
the nucleotide sequence of the B7H3 scFV is SEQ ID NO: 3;
the amino acid sequence of the B7H3 scFV is SEQ ID NO: 4;
the nucleotide sequence of the CD8 hinge region is SEQ ID NO: 5;
the amino acid sequence of the CD8 hinge region is SEQ ID NO: 6;
the nucleotide sequence of the CD28 transmembrane domain is SEQ ID NO: 7;
the amino acid sequence of the CD28 transmembrane domain is SEQ ID NO: 8;
the nucleotide sequence of the 4-1BB intracellular domain is SEQ ID NO: 9;
the amino acid sequence of the 4-1BB intracellular domain is SEQ ID NO: 10;
the nucleotide sequence of the CD3ξ domain is SEQ ID NO: 11; and
the amino acid sequence of the CD3ξ domain is SEQ ID NO: 12.

3. The method for preparing a CAR-T-cell according to claim 1, wherein the method comprises the following steps:
(1) cloning sequences encoding a CD8α leader, a B7H3 scFV, a CD8 hinge region, a CD28 transmembrane domain, a 4-1BB intracellular domain, and a CD3ξ domain into a lentiviral backbone plasmid pCDH-EF1α to construct a recombinant lentiviral plasmid pCDH-EF1α-B7H3-CAR;
(2) co-transfecting the recombinant lentiviral plasmid pCDH-EF1α-B7H3-CAR and a helper plasmid pMD2.G/pSPAX2 obtained in the step (1) into 293T-cells for packaging to obtain a pCDH-EF1α-B7H3-CAR lentivirus;
(3) using the pCDH-EF1α-B7H3-CAR lentivirus obtained in the step (2) to activate and amplify a T-cell cultured for 5 days in vitro to obtain a B7H3-CAR-T-cell;
(4) by CRISPR-Cas9 technology, transferring a ribonucleoprotein (RNP) formed by the ready-to-use sgRNA and a Cas9 protein into the B7H3-CAR-T-cell obtained in the step (3), and specifically targeting and knocking out the human PCSK9 gene to obtain a sgPsck9-B7H3-CAR-T-cell;
the nucleotide sequence of the CD8α leader is SEQ ID NO: 1;
an amino acid sequence of the CD8α leader is SEQ ID NO: 2;
the nucleotide sequence of the B7H3 scFV is SEQ ID NO: 3;
the amino acid sequence of the B7H3 scFV is SEQ ID NO: 4;
the nucleotide sequence of the CD8 hinge region is SEQ ID NO: 5;
the amino acid sequence of the CD8 hinge region is SEQ ID NO: 6;
the nucleotide sequence of the CD28 transmembrane domain is SEQ ID NO: 7;
the amino acid sequence of the CD28 transmembrane domain is SEQ ID NO: 8;
the nucleotide sequence of the 4-1BB intracellular domain is SEQ ID NO: 9;
the amino acid sequence of the 4-1BB intracellular domain is SEQ ID NO: 10;
the nucleotide sequence of the CD3ξ domain is SEQ ID NO: 11; and
the amino acid sequence of the CD3ξ domain is SEQ ID NO: 12.

4. The method for preparing a CAR-T-cell according to claim 2, wherein
in the Step 1, steps for constructing the recombinant lentiviral plasmid pCDH-EF1α-B7H3-CAR comprise:
Step 1-1, performing a double digestion for the lentiviral backbone plasmid pCDH-EF1α with BamHI and NotI restriction enzymes, and recovering a product by a gel extraction;
Step 1-2, mixing a fragment recovered from the Step 1-1 and the fusion gene fragment encoding the CD8α leader, the B7H3 scFV, the CD8 hinge region, the CD28 transmembrane domain, the 4-1BB intracellular domain, and the CD3ξ domain at a molar ratio of 1:1, linking with a T4 DNA ligase, and then transforming into a Stbl3 competent cell; and
Step 1-3, selecting a monoclonal cell of a transformed Stbl3 competent cell for a plasmid extraction, wherein an extracted plasmid is the pCDH-EF1α-B7H3-CAR.

5. The method for preparing a CAR-T-cell according to claim 2, wherein in the Step 2, steps for linking the artificial miRNA are as follows:
Step 2-1, performing a first double digestion for the plasmid pCDH-EF1α-B7H3-CAR obtained in the S1 with BclI and HpaI for a first gel extraction;
Step 2-2, performing a second double digestion for the artificial miRNA with the BclI and the HpaI for a second gel extraction;
Step 2-3, linking a fragment recovered in the Step 2-1 with a fragment recovered from the Step 2-2 by a T4 DNA ligase, and then transforming into a Stbl3 competent cell; and
Step 2-4, selecting a monoclonal cell of a transformed Stbl3 competent cell for a plasmid extraction, wherein an extracted plasmid is the pCDH-EF1α-miRNA-B7H3-CAR.

6. The method for preparing a CAR-T-cell according to claim 2, wherein in the S Step 4, a human T-cell is activated and amplified for 5 days to obtain a cultured human T-cell, and the cultured human T-cell is reinfected with the pCDH-EF1α-miRNA-B7H3-CAR lentivirus.

7. The method for preparing a CAR-T-cell according to claim 3, wherein in the step (4), a gene knocking out process comprises:
delivering an RNP complex into the B7H3-CAR-T-cell by electroporation for gene editing to prepare the sgPack9-B7H3-CAR-T-cell.
